(19) United States
(12) Patent Application Publication
(10) Pub. No.: US 2026/0002922 A1
(43) Pub. Date: Jan. 1, 2026

(54) METHOD OF DETERMINING PHOSPHATE CONCENTRATION IN A WATER SAMPLE

(71) Applicant: HACH LANGE GMBH, Berlin (DE)

(72) Inventors: Heinz Rudde, Berlin (DE); Michael Kussmann, Berlin (DE); Markus Hahn, Berlin (DE); Markus Lenhard, Berlin (DE)

(21) Appl. No.: 18/869,115

(22) PCT Filed: Jun. 1, 2023
(86) PCT No.: PCT/EP2023/064637

§ 371 Date: Nov. 25, 2024

(30) Foreign Application Priority Data
Jun. 1, 2022 (EP) 22176715.5

(51) Int. Cl. G01N 33/18

(52) U.S. Cl. CPC G01N 33/182

(57) ABSTRACT

A method for determining a phosphate concentration in a water sample. The method includes providing the water sample, determining a first phosphate concentration of the water sample via a molybdenum yellow method or via a variant of the molybdenum yellow method at a first temperature, and, if the first phosphate concentration is falls within a specified range, determining a corrected phosphate concentration of the water sample via the molybdenum yellow method or via the variant of the molybdenum yellow method at a second temperature.

The present invention relates to a method for determining phosphate in a water sample.

Attempts have been made for many years to reduce phosphate pollution in the environment, in particular from wastewaters.

According to the German Waste Water Ordinance, water treatment plants having a designed capacity for servicing more than 10,000 inhabitants must adhere to a discharge concentration of phosphate of not more than 2 mg/l. This value is 1 mg/l for a designed capacity for more than 100,000 inhabitants.

An exact determination of phosphate content is therefore necessary.

The molybdenum blue method and the molybdenum yellow method are among the currently most commonly used methods for measuring orthophosphate in a water system, in particular in industrial water. The molybdenum blue method is very sensitive and is suitable for measuring orthophosphate in low concentrations. However, in industrial water system, the orthophosphate concentration may sometimes reach as high as tens of ppm. When the orthophosphate concentration in water is high, the molybdenum blue method is easily saturated so that the water sample must be diluted before the measurement is performed. In contrast to the molybdenum blue method, the molybdenum yellow method has a wide range of measurement and is therefore more suitable for industrial water samples.

The first step of the molybdenum yellow method is to acidify the water sample and to add a molybdate, where the orthophosphate and the molybdate react in the acidic aqueous solution to generate a solution that contains the phosphomolybdic acid (a heteropoly acid). A color development agent, such as vanadate, is then added to the aforesaid phosphomolybdic acid containing solution to develop a yellow color. The absorbance of the water sample is subsequently measured photometrically using a spectrometer, thereby optically determining the concentration of the orthophosphate contained in water.

DE 27 28 706 describes a method for automatic phosphate determination using the molybdenum yellow method which uses as a reagent a solution which contains 25 g of ammonium heptamolybdate, 1 g of ammonium monovanadate, 56 ml of concentrated sulfuric acid, and 1 g of sodium chloride per liter. The molybdate and vanadate components form a yellow-colored complex together with phosphate. It is presumed that the following reaction takes place:

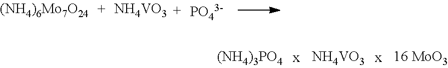

The equilibrium lies on the right-hand side of the equation in an acid medium (pH<2). In this method, a wastewater sample is measured photometrically, the reagent is added, and after a delay time of from 4 to 5 minutes, a second measurement is carried out. The phosphate concentration can be determined from the yellow coloration with the aid of a standard solution. The intensity of the yellow color is here proportional to the phosphate concentration when the measurement solution is strongly acidic.

Variants of the above method are also known.

For example, WO 2016/133882 A1 describes a variant where the composition for measuring the concentration of orthophosphate in a water system is provided in solid form, in particular as a dried powder, in order to facilitate transportation, packaging and use. The composition for measuring the concentration of orthophosphate in a water system comprises:
(a) An acid in solid form;
(b) A water soluble molybdate; and
(c) A color development agent for developing the phosphomolybdic acid.

WO 2016/133882 A1 describes that the color development agent for developing the phosphomolybdic acid is a color development agent used in the molybdenum yellow method that develop a color for the phosphomolybdic acid formed from the reaction between the molybdate and the orthophosphate in the water system under the acidic conditions, for example, vanadates, for example, ammonium vanadate, sodium vanadate and potassium vanadate.

Another variant of molybdenum yellow method is described, for example, in U.S. Pat. No. 4,544,639 which describes a process for determining the amount of organic phosphonate present in an aqueous solution, comprising the steps of:
(a) Adding 1000 to 20,000 ppm of a strong oxidizing agent and 8 ppb to 5000 ppm silver ion to the aqueous solution;
(b) Allowing the organic phosphonate present in said solution to react to form orthophosphate;
(c) Precipitating any excess silver in said solution by the addition of 1000 to 50,000 ppm of a reducing agent or at least an equimolar amount of a halide, based on the silver ion added in Step (a), and removing any precipitate formed;
(d) Adding to said solution 5 to 30 percent, by volume, of an aqueous acid solution containing 500 to 2000 ppm of ions selected from the group consisting of molybdate ion and vanadate ion, and if a halide is used in Step (c), adding 1000 to 50,000 ppm of a reducing agent;
(e) Measuring the absorbance of said solution at 625 to 880 nanometers and determining the orthophosphate concentration from a calibration curve of orthophosphate concentration versus absorbance; and
(f) Subtracting any orthophosphate present in the original aqueous solution from the orthophosphate concentration determined in Step (e), to give the total organic phosphonate concentration.

US 2020/0340925 describes a variant of the molybdenum yellow method which focuses on reliably determining small phosphate concentrations. US 2020/0340925 thereby describes a method for determining phosphate in a water sample comprising:
(a) Providing a water sample;
(b) Adding an acid so that the concentration of the acid in the water sample is at least 0.5 wt.-%;
(c) Performing a first photometric measurement of the water sample solution;
(d) Adding a coloring component to the photometric water sample solution;
(e) Performing a second photometric measurement; and
(f) Calculating the phosphate concentration from the difference between the first photometric measurement and the second photometric measurement.

US 2020/0340925 describes that the coloring reagent, i.e., a reagent based on molybdate and vanadate, is divided by firstly adding an acid before a first photometric measurement is carried out and then adding the coloring component to the same water sample solution and carrying out the second photometric measurement.

Each of DE 27 28 706 A, WO 2016/133882 A1, U.S. Pat. No. 4,544,639 and US 2020/0340925 are incorporated by reference herein. A skilled person would have knowledge of other variants, and the above examples of the molybdenum yellow method is not intended to be limiting.

A disadvantage of the molybdenum yellow method is that inaccuracies have been observed, in particular, when processing a wastewater sample and/or an industrial water sample having a low phosphate content at warm temperatures.

An object of the present invention is to provide an improvement of the molybdenum yellow method to overcome the above inaccuracies.

In an embodiment, the present invention provides a method for determining a phosphate concentration in a water sample. The method includes providing the water sample, determining a first phosphate concentration of the water sample via a molybdenum yellow method or via a variant of the molybdenum yellow method at a first temperature, and, if the first phosphate concentration falls within a specified range, determining a corrected phosphate concentration of the water sample via the molybdenum yellow method or via the variant of the molybdenum yellow method at a second temperature.

In an embodiment of the present invention, if the specified range of the first phosphate concentration is <5 ppm, the second temperature for the corrected phosphate concentration is preferably 30-35° C., if the specified range of the first phosphate concentration is from 5-10 ppm, the second temperature for the corrected phosphate concentration is preferably 35-40° C., and if the specified range of the first phosphate concentration is from 10-15 ppm, the second temperature for the corrected phosphate concentration is preferably 40-44° C.

In an embodiment of the present invention, the first temperature is preferably from 40-60° C., more preferably from 43-47° C., and most preferably at approximately 45° C. The first temperature can, for example, be 40° C., 41° C., 42° C., 43° C., 44° C., 45° C., 46° C. 47° C., 48° C., 49° C., 50° C., 51° C., 52° C., 53° C., 54° C., 55° C. 56° C., 57° C., 58° C., 59° C. or 60° C.

In an embodiment of the present invention, the water sample is preferably divided into two water samples, for example, a first water sample and a second water sample. The determining of the first phosphate concentration is preferably performed with one of the two water samples, for example, with the first water sample, while the determining of the second phosphate concentration is performed with the other of the two water samples, for example, with the second water sample.

In an embodiment of the present invention, the determining of the first phosphate concentration and the determining of the second phosphate concentration is performed by adjusting the temperature of the water sample from the first temperature to the second temperature. A skilled person knows that the molybdenum yellow method will take a certain amount of time, for example, up to five minutes. Instead than running two samples, which would then up to 10 minutes, excluding any preparation time, the skilled person can instead use a single sample and perform the analysis with an analysis system where the single sample can be heated and/or cooled in situ. Analysis systems with heating and/or cooling systems have previously been described, for example, in U.S. Pat. Nos. 4,004,708, 4,619,530 or in CN 104316479 A, each of which is incorporated by reference herein. An aspect of the present invention therefore includes an analyzer having a flexible photometric reaction chamber with a flexible temperature range of 25-60° C. which can be heated and/or cooled between any temperature from 26-60° C. within a matter of seconds. This practically immediate temperature adjustment allows a photometric measurement to be conducted first at the first temperature, and almost immediately thereafter, at the second temperature, with the same sample. A skilled person could, merely as an example, perform a first analysis at 45° C., and a second analysis at 30° C. Any difference in the analysis result could thereby be the result of an interference. The use of two temperatures could therefore also be used by a skilled person to check and/or validate the analysis result obtained at another temperature. As stated above, any combination of temperatures between 25 and 60° C. is possible (provided that the starting temperature is not equal to the ending temperature), for example:

| | |
|---|---|
| Starting | Ending |
| Temperature | Temperature |

| | | | | | | |
|---|---|---|---|---|---|---|
| 25 | 37 | 49 | Combined | 25 | 37 | 49 |
| 26 | 38 | 50 | With | 26 | 38 | 50 |
| 27 | 39 | 51 | | 27 | 39 | 51 |
| 28 | 40 | 52 | | 28 | 40 | 52 |
| 29 | 41 | 53 | | 29 | 41 | 53 |
| 30 | 42 | 54 | | 30 | 42 | 54 |
| 31 | 43 | 55 | | 31 | 43 | 55 |
| 32 | 44 | 56 | | 32 | 44 | 56 |
| 33 | 45 | 57 | | 33 | 45 | 57 |
| 34 | 46 | 58 | | 34 | 46 | 58 |
| 35 | 47 | 59 | | 35 | 47 | 59 |
| 36 | 48 | 60 | | 36 | 48 | 60 |

In an embodiment of the present invention, the water sample is preferably a wastewater sample or an industrial water sample. A skilled person knows that such samples are usually photometrically analyzed at approximately 45° C.

The corrected phosphate concentration preferably mitigates an effect of an interference of the phosphate concentration at this temperature.

The interference is preferably a silicate interference. The term “silicate” is thereby understood to be any member of a family of anions consisting of silicon and oxygen, usually with the general formula

$${[ {SiO}_{4 - x}^{( 4 - 2 x ) -} ]}_{n}$$

where 0≤x<2. The family includes orthosilicate

$${SiO}_{4}^{4 -} ( x = 0 ) ,$$

metasilicate

$${SiO}_{3}^{2 -} ( x = 1 ) ,$$

and pyrosilicate

$${Si}_{2} {O}_{7}^{6 -} ( x = 0.5 , n = 2 ) .$$

The name is also used for any salt of such anions, such as sodium metasilicate; or any ester containing the corresponding chemical group, such as tetramethyl orthosilicate. The term silicate is used to mean silicate minerals, ionic solids with silicate anions, as well as rock types that consist predominantly of such minerals. In that context, the term also includes the non-ionic compound silicon dioxide SiO₂ (silica, quartz), which would correspond to x=2 in the general formula. The term also includes minerals where aluminum or other tetravalent atoms replace some of the silicon atoms, as in the aluminosilicates. The interference of the present invention is of course not limited to silicate, other examples of interference are also known to the skilled person, for example, Zr(IV) and Ti(IV), Ta(V) and Nb(V), W(VI).

A variant of the method for determining a phosphate concentration in a water sample of the present invention includes providing the water sample, determining a first phosphate concentration of the water sample via a molybdenum yellow method or via a variant of the molybdenum yellow method at a temperature of from 40-50° C., preferably from 43-47° C., very preferably at approximately 45° C., determining a silicate concentration of the water sample, and correcting the first phosphate concentration by applying a phosphate correction value so as to eliminate an interference effect of the silicate concentration.

A skilled person would know many methods of determining the silicate concentration of a water sample. One such method is described in ASTM D859-16 (2021) e1. Another method is the Spectroquant® Silicate Test 101813 of Merck KGaA which starts by pipetting 20 ml of sample solution into a plastic test vessel, after which 200 μL of a reagent (Si-1) is added. The solution is then mixed and then left to stand for 5 minutes. After the standing time, 200 μL a second reagent (Si-2) is added and the solution is mixed, then 1.00 mL of a third reagent (Si-3) is added. The solution is mixed once again, left to react for 5 minutes, and then measured in the photometer against a reagent blank prepared with Ultrapure water in an analogous manner. Another method is the “Silicomolybdate/Heteropoly Blue Method” of Hach Lange GmbH. The Silicomolybdate Method involves the reaction of molybdate ion with silica and phosphate under acid conditions to form a yellow color. Citric acid is added to destroy the phosphomolybdic acid complex (the yellow color formed due to phosphate), but not the silicomolybdic acid complex. For large amounts of silica, the remaining yellow color is intense enough to be read directly. For low concentrations, an amino-naphthol sulfonic acid reducing agent is used to convert the faint yellow color to a dark heteropoly blue species. The color formed is directly proportional to the amount of silica present in the original sample; a colorimetric measurement of this intensity provides an accurate means of determining the silica concentration. Some forms of silica (usually polymeric) will not react with ammonium molybdate and must be digested with sodium bicarbonate to be converted to a reactive form. Silicic acid reacts with water and hydrates as follows:

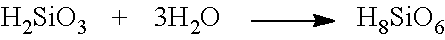

This hydrated silicic acid reacts with molybdate in the presence of acids to form silicomolybdic acid.

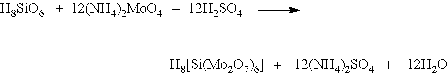

This silicomolybdic acid is then reduced to a blue color (heteropoly species) by an amino naphthol sulfonic acid for low concentrations. The above examples are not intended to be limiting and a skilled person would have knowledge of additional methods.

In an embodiment of the present invention, the phosphate correction value is preferably,
a) from 0 to 25 ppm of silicate: (the determined ppm silicate)×0.10/(25 ppm silicate),
b) from >25 to 50 ppm of silicate: additionally to a), (the determined ppm silicate exceeding 25 ppm)×0.07/(25 ppm silicate), and
c) from >50 to 100 ppm of silicate: additionally to a) and b), (the determined ppm silicate exceeding 50 ppm)×0.20/(50 ppm silicate).

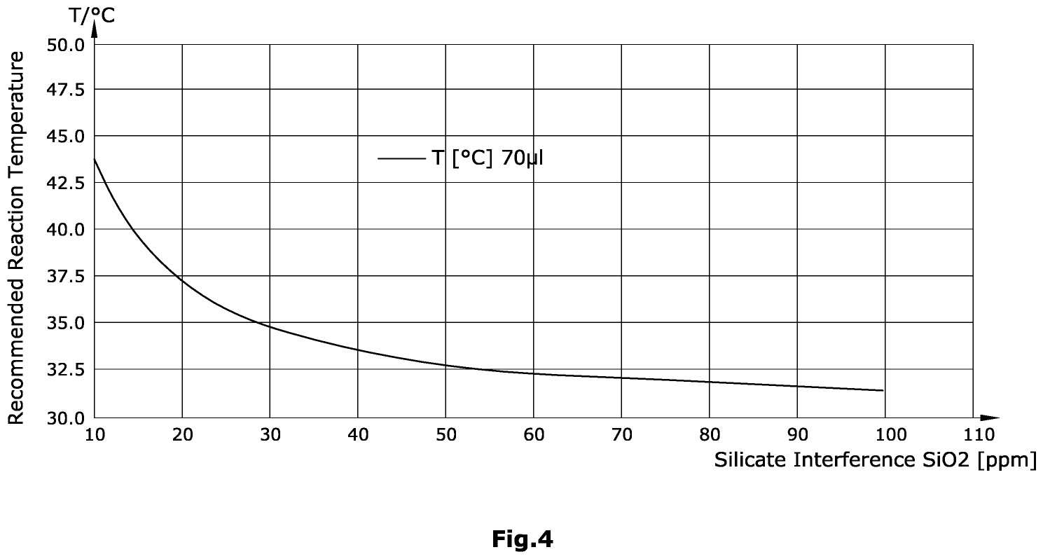

The above correction values are merely provided as an example. A skilled person can calculate narrower or broader ranges based on the water sample usually used. In an embodiment of the present invention, the phosphate correction value can also be provided by a look-up table and/or an assumption modeling which represents the interference effect of the silicate concentration at a specific temperature. An example of an assumption modeling is shown, for example, in FIG. 4. A skilled person would then deduct this interference effect from the first phosphate concentration based on the silicate concentration of the water sample determined, for example, in ppm silicate, and temperature at which the first phosphate concentration was determined. The corrected first phosphate concentration will then be obtained absent the interference effect of the silicate concentration.

A variant of the method for determining a phosphate concentration in a water sample of the present invention is to eliminate the interference effects of silicate by always determining the phosphate concentration at a temperature where no, or at least minimal, such interference effects occur. This variant preferably provides the water sample, establishes a temperature of the water sample of preferably 30-35° C., and then determines the phosphate concentration of the water sample via the molybdenum yellow method or a variant thereof. The temperature of the water sample can, for example, even be lower than 30° C., for example, from 10-30° C. Preference is however given to a temperature of 30° C., 31° C., 32° C., 33° C. and 35° C., for example, 30° C.

The present invention is described in greater detail below on the basis of embodiments and of the drawings in which:

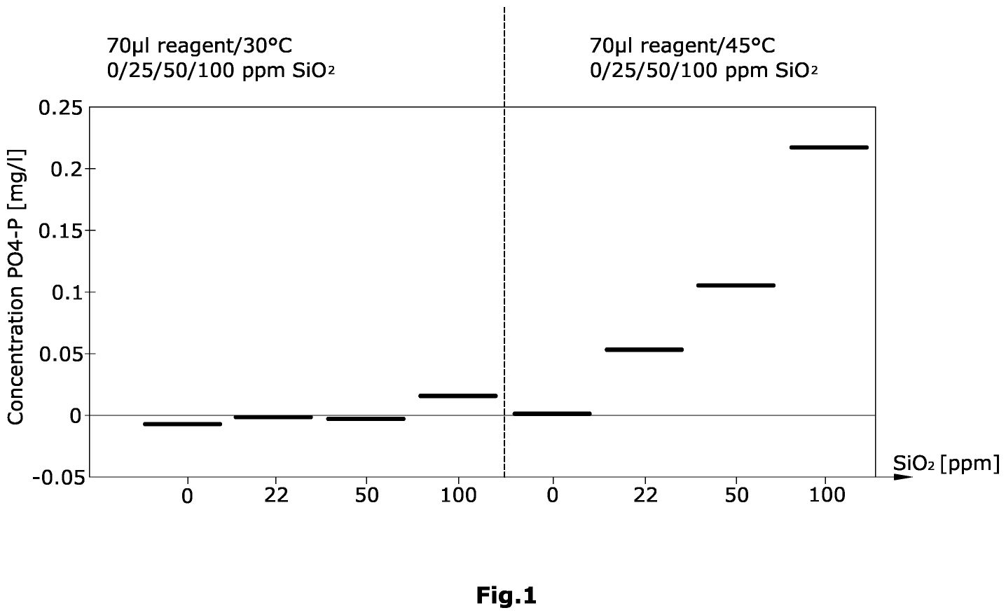

FIG. 1 shows the optimizing of silicate interference at temperatures of 30° C. and 45° C.

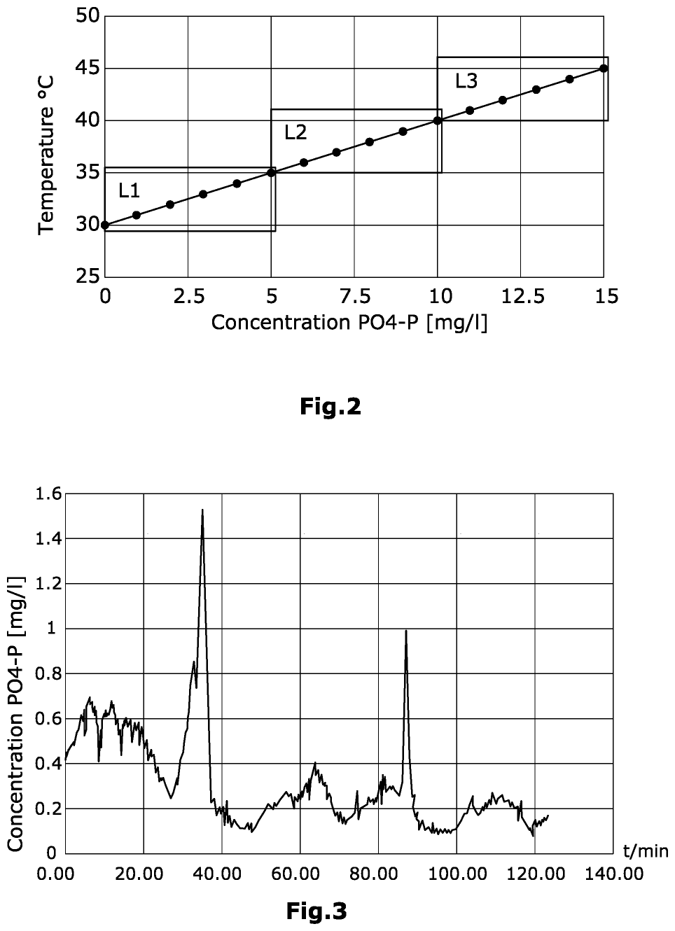

FIG. 2 shows an algorithm for forecasting a result of an increasing/decreasing phosphate concentration slope.

FIG. 3 shows a typically run of a wastewater plant in the range of <5 mg/l PO₄-P over time.

FIG. 4 shows an assumption modeling between reaction temperature and silicate interference.

The present invention is explained in greater detail below based on the following examples which are provided to show how the present invention can be practically applied in the real world. The examples are not intended to be limiting.

### EXAMPLES

### Example 1

In order to investigate the impact that silicate (here SiO₂) has on an orthophosphate measurement, a sample run was performed under standard conditions using the Phosphax sc Phosphat-Analysator from Hach Lange GmbH, 0.05-15 mg/l PO₄-P (55 μl reagent to 1 ml sample at 45° C. and less than 5 Minutes reaction time) with a silicate range of 0-100 mg/l SiO₂. It was thereby determined that the interference of silicate can result in up to 0.37 ppm phosphate equivalents as PO₄-P with 100 ppm silicate. Table 1 sets forth the data of samples run between 0-100 ppm SiO₂.

TABLE 1

| | | ppm PO4—P |
|---|---|---|
| Sample | ppm SiO₂ | equivalent |
| 1 | 0 | 0.00 |
| 2 | 25 | 0.10 |
| 3 | 50 | 0.17 |
| 4 | 100 | 0.37 |

### Example 2

A first sample run was repeated under optimized conditions using the Phosphax sc Phosphat-Analysator from Hach Lange GmbH, 0.05-15 mg/l PO₄-P (70 μl reagent to 1 ml sample at 45° C. and less than 5 Minutes reaction time) with a silicate range of 0-100 mg/l SiO₂. The only difference between Examples 1 and 2 was therefore that more reagent (70 μl instead of 55 μl) was used. It was thereby determined that the interference of silicate can result in up to 0.22 ppm phosphate equivalents as PO₄-P with 100 ppm silicate. Table 2 sets forth the data of samples run between 0-100 ppm SiO₂.

TABLE 2

| | | ppm PO4—P |
|---|---|---|
| Sample | ppm SiO₂ | equivalent |
| 1 | 0 | 0.00 |
| 2 | 25 | 0.05 |
| 3 | 50 | 0.11 |
| 4 | 100 | 0.22 |

The interference impact could therefore be reduced by up to 40% by manipulating the sample-to-reagent ratio.

### Example 3

Example 2 was repeated at a temperature of 30° C. instead of 45° C. It was thereby determined that the interference of silicate can be significantly reduced. Table 3 sets forth the data of samples run between 0-100 ppm SiO₂. A comparison of the results of Examples 2 and 3 are set forth in FIG. 1. FIG. 1 thereby clearly shows that the interference of silicate is significant at higher temperatures (i.e., 45° C.) compared to lower temperatures (i.e., 30° C.).

TABLE 3

| | | ppm PO4—P | ppm PO4—P |
|---|---|---|---|
| | | equivalent | equivalent |
| Sample | ppm SiO₂ | at 45° C. | at 30° C. |
| 1 | 0 | 0.00 | −0.001 |
| 2 | 25 | 0.05 | 0.000 |
| 3 | 50 | 0.11 | 0.000 |
| 4 | 100 | 0.22 | 0.015 |

Examples 2 and 3 shows that the interference of silicate can be corrected based on a combination of temperature and sample-to-reagent ratio to approximately 93-100%.

The above Examples demonstrate that varying only the ratio of sample-to-reagent will not in and of itself significantly minimize or entirely eliminate silicate interference. The above Examples also show, however, that changing the reaction temperature, for example, in the range of 30-60° C., can significantly minimize or entirely eliminate silicate interference. While the present invention does not propose any conclusive underlying theory for this technical effect, it is believed that reaction kinetics may be influenced based on the Arrhenius equation in combination with the chosen concentration level. A practical application of this observation thereby involves dividing the measuring range into segmented reaction temperature areas or zones to thereby minimize or entirely eliminate silicate interference. For example, 50 mg/l of silicate showed an impact of 0.17 mg/l phosphate under standard conditions at 45° C. (see Example 1 above). A reduction of the temperature to 30° C. reduced this interference entirely (see Example 3 above). An analysis with high resolution down to 0.05 mg/l is therefore possible.

It is also possible to run an assumption modeling for various phosphate concentrations. FIG. 4 shows, for example, an assumption modeling between reaction temperature and silicate interference based on a reagent ratio of 70 μl to 1 ml sample. FIG. 4 thereby shows that the effect of silicate interference is reduced when the temperature is lowered. A skilled person can use such an assumption modeling to choose a reaction temperature with an expected silicate interference in order to minimize the impact of SiO₂.

1-10. (canceled)

11. A method for determining a phosphate concentration in a water sample, comprising:
providing the water sample;
determining, using a molybdenum yellow method, a first phosphate concentration of the water sample at a first temperature; and
determining, using the molybdenum yellow method, responsive to determining the first phosphate concentration falls within a specified range, a corrected phosphate concentration of the water sample at a second temperature.

12. The method of claim 11, wherein when the specified range of the first phosphate concentration is less than 5 ppm, the second temperature for the corrected phosphate concentration is 30-35° C.

13. The method of claim 11, wherein when the specified range of the first phosphate concentration is between 5-10 ppm, the second temperature for the corrected phosphate concentration is 35-40° C.

14. The method of claim 11, wherein when the specified range of the first phosphate concentration is between 10-15 ppm, the second temperature for the corrected phosphate concentration is 40-44° C.

15. The method of claim 11, wherein the first temperature is between 40-60° C.

16. The method of claim 11, wherein the water sample is divided into a first water sample and a second water sample, and the determining of the first phosphate concentration is performed with the first water sample and the determining of the second phosphate concentration is performed with the second water sample.

17. The method of claim 11, wherein the determining of the first phosphate concentration and the determining of the second phosphate concentration is performed by adjusting the temperature of the water sample from the first temperature to the second temperature.

18. The method of claim 11, wherein the water sample is selected from the group consisting of: a wastewater sample and an industrial water sample.

19. The method of claim 11, wherein the corrected phosphate concentration mitigates an effect of an interference of the first phosphate concentration.

20. The method as recited in claim 19, wherein the interference is a silicate interference.

21. A method for determining a phosphate concentration in a water sample, comprising:
providing the water sample;
determining, using a molybdenum yellow method, a first phosphate concentration of the water sample at a temperature of from 40-50° C.;
determining a silicate concentration of the water sample; and
correcting the first phosphate concentration by applying a phosphate correction value to eliminate an interference effect of the silicate concentration.

22. The method of claim 21, wherein the phosphate correction value is, for between 0 to 25 ppm of silicate, (the determined ppm silicate)×0.10/(25 ppm silicate).

23. The method of claim 21, wherein the phosphate correction value is, for between 25 to 50 ppm of silicate, (the determined ppm silicate exceeding 25 ppm)×0.07/(25 ppm silicate).

24. The method of claim 21, wherein the phosphate correction value is, for between 50 to 100 ppm of silicate, (the determined ppm silicate exceeding 50 ppm)×0.20/(50 ppm silicate).
